(19) United States
(12) Patent Application Publication
(10) Pub. No.: US 2020/0002244 A1
(43) Pub. Date: Jan. 2, 2020

(54) Process for Converting Butanol into Propylene

(71) Applicant: TOTAL RESEARCH & TECHNOLOGY FELUY, Seneffe (BE)

(72) Inventors: Nikolai Nesterenko, Nivelles (Thines) (BE); Walter Vermeiren, Houthalen (BE); Delphine Minoux, Nivelles (BE); Jean-Pierre Dath, Beloeil (BE)

(21) Appl. No.: 16/061,632

(22) PCT Filed: Dec. 14, 2016
(86) PCT No.: PCT/EP2016/080930

§ 371 Date: Jun. 12, 2018

(30) Foreign Application Priority Data
Dec. 14, 2015 (EP) 15199865.5

(51) Int. Cl. C07C 1/22; B01J 23/44; B01J 23/89; B01J 21/08; B01J 37/02; B01J 37/08

(52) U.S. Cl. CPC C07C 1/22; B01J 23/44; B01J 23/892; B01J 21/08; B01J 37/0207; B01J 37/08; C07C 2523/50; C07C 2527/25; C07C 2523/44; C07C 2523/89; C07C 2523/755; C07C 2521/08; B01J 37/0236

(57) ABSTRACT

Process for selective the conversion of primary C4 alcohol into propylene comprising: contacting a stream (1) containing essentially a primary C4 alcohol with at least one catalyst at a temperature ranging from 150° C. to 500° C. and at pressure ranging from 0.01 MPa to 10 MPa conditions effective to transform said primary C4 alcohol into an effluent stream (2, 5) containing essentially propylene, carbon monoxide and di-hydrogen, said transformation of primary C4 alcohol comprising at least a reaction of decarbonylation and optionally a decarboxylation reaction, said at least one catalyst comprising a support being a non-acidic i.e. having a TPD NH3 of less than 50 preferably less than 40 μmol/g and optionally a non-basic catalyst i.e. having a TPD CO2 of less than 100 preferably less than 50 μmol/g.

### FIELD OF THE INVENTION

Propylene used to prepare polymers is traditionally produced in a naphtha steam cracker. It is also a by-product of the FCC (Fluid Catalytic Cracking) refinery unit. There is an increase of the propylene demand that tends to outpace ethylene demand. Simultaneously, development of cheap ethane cracked in steam crackers irreversibly leads to the lightening of cracking feedstock's and consequently to an increased production of ethylene versus propylene. Considering additionally the environmental concerns and the limited supply and increasing cost of crude oil, it appears relevant to prepare propylene from bio sourced molecules. More and more companies offer technologies for the production of bio butanol via for instance the retrofitting of bio ethanol plants to bio butanol production or via the selective production of iso butyl alcohol from synthesis gas. Butanol is therefore an available bio sourced molecule that can be further transformed into valuable products such as propylene.

The present invention is a process for converting butanol, preferably iso-butanol originating from a bio source, into propylene. In particular, the process involves in one of its step the decarbonylation and/or the decarboxylation reaction. The formation of the olefins is achieved via reduction of the carbon chain length and production of di-hydrogen (H₂) and carbon monoxide (CO) and/or carbon dioxide (CO₂).

### BACKGROUND OF THE INVENTION

U.S. Pat. No. 3,578,423 discloses a process for catalytically splitting iso-butyraldehyde in presence of steam to produce essentially carbon monoxide and hydrogen, In this process, iso-butyraldehyde and water vapor in amount of at least 3 moles water vapor per g-atom aldehyde carbon are reacted at temperature of about 600-900° C., at ordinary atmospheric or elevated pressure with a nickel containing catalyst.

U.S. Pat. No. 3,535,400 discloses a process for thermally splitting iso-butyraldehyde to form a gas mixture composed essentially of propylene, carbon monoxide and hydrogen. The process comprises heating iso-butyraldehyde at a temperature in the range of 500 to 800° C. in the presence of steam.

U.S. Pat. No. 4,200,589 discloses a process for the preparation of acetone by the catalytic oxidative decarbonylation of iso-butyraldehyde in the gaseous phase by passing a mixture consisting of iso-butyraldehyde, oxygen and an inert diluent, when desired, at higher temperatures and at contact times from 0.1 to 10 seconds over a carrier catalyst wherein the catalyst contains a mixture of copper oxide or manganese oxide, or a mixture thereof, with zinc oxide. The process is conducted at a temperature of about 130° C. to 180° C.

U.S. Pat. No. 3,352,924 describes the oxo synthesis (the carbonylation reaction) which is the reverse reaction of the reaction of decarbonylation. It relates to the preparation of oxygenated organic compounds by the reaction of carbon monoxide and hydrogen with carbon compounds containing olefinic linkages in the presence of a carbonylation catalyst. The reaction is performed at a temperature in the range of 66° C. (150° F.) to 232° C. (450° F.) and a pressure in the range of 3.4 MPa (500 psig) to 34 MPa (5000 psig) in the presence of a crystalline metallic alumina-silicate catalyst.

U.S. Pat. No. 4,262,157 deals with the decarboxylation of carboxylic acids using diazabicycloalkenes and optionally copper salts at a temperature of 0° C. to 400° C. for a period of 0.1 to 50 hours. The reaction is performed by reaction of one molar equivalent of carboxylic acid with 1 to 3 molar equivalents of diazabicycloalkene.

U.S. Pat. No. 4,039,584, discloses a process to improve the yield of n-butyraldehyde in propylene carbonylation (oxo-synthesis) by separating the n- and iso-butyraldehydes and subjecting to the decarbonylation reaction the iso-butyraldehyde. The iso-butyraldehyde is cleaved to form propylene, carbon monoxide and hydrogen over a supported rhodium or platinum catalyst at a temperature of 200° C. to 400° C. and a pressure of 0.01 MPa (0.1 atmosphere) to 2 MPa (20 atmospheres). The propylene and the gases resulting from the cleavage are returned to the oxo synthesis step to increase the yield of n-butyraldehyde.

U.S. Pat. No. 4,517,400 discloses a use of the decarbonylation reaction to separate i-butyraldehyde and n-butyraldehyde due to different reactivities. The faster decarbonylation rate of the n-butyraldehyde was used to convert it back to propylene and recycle in oxo-synthesis (propylene carbonylation reaction). By this way, the yield of i-butyraldehyde in carbonylation of the propylene was increased. Decarbonylation of n-butyraldehyde to propylene is obtained by passing the aldehydes over a catalyst comprising a metal selected from the group consisting of palladium, platinum, rhodium, copper, silver, gold and zinc supported on or exchanged onto a zeolite. The metal zeolite catalysts exhibit a high selectivity and activity for the decarbonylation of predominantly an n-aldehyde from a reaction mixture of n-aldehyde and a branched aldehyde to produce mainly an olefin, carbon monoxide and hydrogen which can be recycled for the production of iso-aldehydes.

DE 19 13 198 discloses a process for the production of 2-ethyl hexanol from propylene and carbon monoxide in which iso butyraldehyde formed as an intermediate product is decomposed into propylene for being recycled.

US 2014/0088326 discloses a process for the production of higher aldehydes from lower alcohols using a two stage vapor phase heterogeneous catalyst system with a particular first step consisting in the conversion of butanol into butanal.

WO 2012/016787 discloses the conversion of butanol into propylene via the simultaneous dehydration and cracking over a phosphorous modified zeolite.

Selective decomposition of iso-butyraldehyde to propylene, carbon monoxide and hydrogen has also been described by Kaoru Fujimoto (Applied catalysis, 29 (1987) 203-210). Fujimoto reported iso-butyraldehyde is decomposed selectively to carbon monoxide, propylene and hydrogen over a supported palladium catalyst modified by sodium sulphide at a temperature from 150 to 350° C. It is also reported that added sulphide ion effectively suppressed the secondary reaction of propylene hydrogenation.

None of the prior art document discloses the on-purpose transformation of butanol into propylene. They are focused only on a use of decarbonylation of the aldehyde to adjust the selectivity in oxo-synthesis (propylene carbonylation) or steam-assisted catalytic conversion of iso-butyraldehyde above 600° C. The catalyst used in decarbonylation contains acid or basic sites (metal containing zeolites). This type of materials presents the following drawbacks: they present a high dehydration activity or they may catalyse a condensation between iso-butanol and iso-butyraldehyde to heavy products, which is not desirable in the present invention. The present invention on the contrary discloses the transformation of butanol (preferably bio sourced) into propylene. More precisely, it has been discovered that transformation of butanol is possible with a high selectivity to propylene and reduced amount of by products.

### BRIEF SUMMARY OF THE INVENTION

The present invention provides a process for selective conversion of primary C4 alcohol into propylene, comprising:
contacting a stream (1) containing essentially primary C4 alcohol with at least one catalyst at a temperature ranging from 150° C. to 500° C. and at pressure ranging from 0.01 MPa to 10 MPa said conditions being conditions effective to transform said primary C4 alcohol into an effluent stream (2, 5) containing essentially propylene, carbon monoxide and di-hydrogen, said transformation of primary C4 alcohol comprising at least a reaction of decarbonylation and optionally a decarboxylation reaction, said at least one catalyst comprising a support being a non-acidic (i.e. having a TPD NH3 of less than 50 preferably less than 40 μmol/g) and a non-basic (i.e. having a TPD CO2 of less than 100 preferably less than 50 μmol/g) support or said at least one catalyst being preferably a non-acidic (i.e. having a TPD NH3 of less than 50 preferably less than 40 μmol/g) and a non-basic (i.e. having a TPD CO2 of less than 100 preferably less than 50 μmol/g) catalyst.

The term “primary C4 alcohol” shall be understood as referring to primary butanol, that is normal butanol (n butanol) and iso-butanol.

In a particular embodiment, stream (1) essentially contains iso-butanol.

In a particular embodiment, the process is remarkable in that said effluent stream (2,5) at least 1 wt %, preferably 10 wt %, most preferably 25 wt % of primary C4 alcohol is converted into propylene, carbon monoxide and di-hydrogen. The transformation of butanol, especially iso-butanol, to propylene, carbon monoxide and di-hydrogen may occur in one or two reaction zones on the same or different catalyst(s). The non-converted products from both reaction zones can optionally be recycled back.

In a preferred embodiment, the transformation is performed in two reaction zones, where butanol, especially iso-butanol is present in concentration of less than 10 wt % in the second reaction zone.

The term “butyraldehyde” shall be understood as referring to any aldehyde in C4, more precisely any compounds selected from the list comprising butanal and 2-methyl propanal (or iso-butyraldehyde) or any combination of those isomers.

The product of the propylene hydrogenation, i.e. propane, may also be a by-product of the reaction.

Butyric acid could be formed in the reaction due to over oxidation of the C4 alcohol. However, this product may also converted in propylene via a decarboxylation reaction. The term “butyric acid” shall be understood as referring to any carboxylic acid in C4, more precisely any compounds selected from the list comprising 1-propanecarboxylic acid and 2-methylpropanoic acid or any combination of those isomers.

Decarbonylation shall be understood as the transformation of an oxygenated hydrocarbon into an olefin which contains one carbon atom less. The transformation accompanies with CO and H₂ formation. This reaction is different from the normal cracking described, for example, in WO2012016785. The production of the lower olefins by cracking occurs via the consecutive dehydration of the “butanol” to butenes followed by a cracking of the butenes to propylene and ethylene.

In case of the decarbonylation, it has been discovered the reaction takes place via the dehydrogenation/partial oxidation of the butanol to butyraldehyde intermediate and substantially leads to formation of only propylene as the olefin. So, the process allows efficiently converting of the butanol to propylene without making ethylene.

For example the decarbonylation reaction of iso-butanol is presented below:

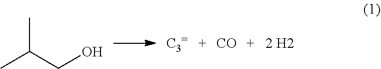

The decarbonylation reaction of iso-butyraldehyde is presented below:

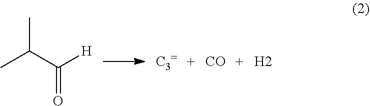

Decarboxylation shall be understood as the transformation of a carboxylic acid or its corresponding salts or soap into an olefin which contains one carbon atom less with CO₂. The decarboxylation reaction of iso-butyric acid is presented below:

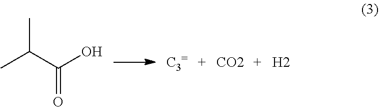

The formed butanoic acid can be also transformed to propylene via decarbonylation in presence of hydrogen though formation of the butyraldehyde intermediate. The hydrogen is naturally present in the system because of the decarbonylation. So, the butanoic acid may be co-processed with butyraldehyde.

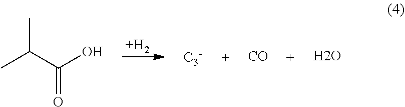

Advantageously, the process for selective conversion of primary C4 alcohol comprises contacting the primary C4 alcohol stream (1) with the said at least one catalyst being preferably non-acidic, which optionally is also a non-basic catalyst, at conditions effective to produce an effluent stream (2, 5) wherein at least 1 wt %, preferably at least 10 wt %, most preferably at least 25 wt % of primary C4 alcohol is converted into propylene, carbon monoxide and di-hydrogen. Optionally unconverted compounds can be separated and recycled into stream (1).

As already explained, the transformation of butanol, especially iso-butanol, to propylene, carbon monoxide and di-hydrogen may occur in one or two reaction zones. Without wishing to be bound by any theory, in case of performing the reaction in a single reaction zone, low basicity preferably of the support of the catalyst or of catalyst may contribute to avoid side-reaction between non-converted butanols and aldehydes. Low acidity may contribute to avoid dehydration of butanol to corresponding olefins and ethers. In case of operating in two reaction zones, a low basicity preferably of the support of the catalyst or of catalyst may be useful only in the first reaction zone. The second reaction zone may use catalysts which contain basic sites. On the contrary, low acidity of preferably of the support of the catalyst or the catalyst may be advantageous for both reaction zones.

In a first embodiment, in the process for selective conversion of primary C4 alcohol, the step of contacting the primary C4 alcohol stream (1) with at least one non acidic preferably catalyst support or catalyst, which is also a non-basic preferably catalyst support or catalyst performed in a single reaction zone (A).

Advantageously, before said transformation of primary C4 alcohol, said non-acidic preferably catalyst support or catalyst, which is also a non-basic preferably catalyst support or catalyst, may be submitted to a reduction in presence of di-hydrogen (H₂) at a temperature of at least 400° C. for at least 1 hour. This may favor the transformation into propylene.

In this first embodiment at least one diluent may optionally be added to stream (1) and optionally said diluent may be recovered in said effluent stream (2) and optionally recycled in stream (1).

The reaction zone (A) can be operated under a temperature ranging from 150° C. to 500° C. preferably ranging from 200° C. to 450° C. and under pressure ranging from 0.01 MPa (0.1 bar) to 10 MPa (100 bars) preferably ranging from 0.1 MPa (1 bar) to 3.5 MPa (35 bars), most preferably from 0.12 MPa (1.2 bar) to 1.5 MPa (15 bars).

In a second embodiment, the process for selective conversion of primary C4 alcohol comprises the following steps:
Contacting stream (1) with at least one first catalyst comprising a support being non-acidic i.e. having a TPD NH3 of less than 50 preferably less than 40 μmol/g and a non-basic i.e. having a TPD CO2 of less than 100 preferably less than 50 μmol/g or preferably one first non acidic catalyst, in a first reaction zone (B) at conditions effective to produce being preferably at a temperature ranging from 150° C. to 500° C. and at pressure ranging from 0.01 MPa to 10 MPa a stream (3) in which at least 1 wt % of primary C4 alcohol is converted into butyraldehyde,
optionally separating unconverted compounds (unconverted primary C4 alcohol) from stream (3) and recycling them into stream (1) to obtain a purified stream (4),
contacting stream (3) and/or optionally the purified stream (4) with at least one second catalyst comprising a support being non-acidic i.e. having a TPD NH3 of less than 50 preferably less than 40 μmol/g and a non-basic i.e. having a TPD CO2 of less than 100 preferably less than 50 μmol/g or preferably a non-acidic catalyst, in a second reaction zone (C) at conditions effective to produce an effluent stream (5) being preferably a temperature ranging from 150° C. to 500° C. and at pressure ranging from 0.01 MPa to 10 MPa comprising essentially propylene, carbon monoxide and di-hydrogen, said second non-acidic catalyst being optionally reduced, before said transformation of stream (3) and/or stream (4), in presence of di-hydrogen at a temperature of at least 400° C. for at least 1 hour,
optionally separating unconverted compounds from effluent stream (5) and recycling them into stream (4) and/or stream (1),

In this second embodiment at least one diluent is optionally added to stream (1) and
optionally said diluent is recovered from said stream (3) and optionally recycled in stream (1),
optionally said diluent is recovered from said effluent stream (5) and optionally recycled in stream (1) and/or stream (3),

Recycling of diluents, if any, can optionally be performed together with recycling of unconverted compounds.

Advantageously, the stream (3) and/or optionally the purified stream (4) treated in the second reaction zone (C) may present a content of unconverted compounds (primary C4 alcohol, in particular iBuOH) of less than 10 wt %, preferably of less than 5%, most preferably of less than 1 wt %. This may permit to avoid the formation of secondary products, in particular heavies, in the second reaction zone (C).

The first and second catalyst(s) in the reaction zones B and C may be the same or different. Advantageously, only the second catalyst used in the second reaction zone C is submitted to a reduction prior to reaction. This may favor the formation of propylene and stable performance.

In this second embodiment an oxidant may optionally be added to stream (1) and
optionally said oxidant is recovered from said stream (3) and optionally recycled in stream (1)
the said oxidant could be present or not in the stream (3).

Preferably, the oxidant is not present in the stream (3).

The preferred oxidant is air.

Advantageously, the non-acidic catalyst, which optionally may also be non-basic, comprises at least one non-acidic support, which optionally is also a non basic support, and optionally comprises at least one metal dispersed on said at least one support at a content of at least 0.05 wt % based on the weight of the catalyst.

In another embodiment, the non-acidic and optionally non-basic catalyst consists of least one metal. The at least one metal may be non-supported, for example in the form of wire gauze or bulk crystals, and may be in a metallic state, in an oxidized state or in a partially reduced oxide state.

Whatever the reaction zone, the above catalyst(s) may comprise one or several of the following characteristics
at least one metal is a transition metal preferably chosen in the groups IB, IIB, IVB, VB, VIB, VIIIB, most preferably palladium and/or platinum,
at least one metal is chosen in the groups IIIA and IVA,
two metals chosen in the groups IB, IVB, VB, VIB, VIIIB, IIIA, IVA, for example selected from CoMo, MoFe, NiW, NiMo, Cu—Pd, Cu—Ni, Cu—Co, Cu—Pt, Fe—Pd, Co—Pd, Ni—Pd, Pt—Pd, Ag—Pd, Au—Pd, Fe—Pt, Ni—Pt, Pt—Sn, Pt—Pb, Pd—Sn, Pd—Pb, Au—Pd, Nb—Pd, Ga—Pd, Zr—Pd,
said at least one non-acidic and optionally non-basic support is selected from passivated alumina, activated carbon, metal silicate, perovskytes, silica-magnesia, phalocianides, silica, ceria, zirconia, titania, clays.

Advantageously, said non-acidic and optionally non-basic catalyst or support may comprise, or may consist of, activated carbon, silica, or a mixture of thereof. In general, the non-acidic and optionally non-basic catalyst or support may advantageously contain a content of alkali cations (Na, K) to neutralize the residual acidity, phosphorous, boron and/or S, each at a content of less than 2 wt % based on the weight of the catalyst, to limit the hydrogenation activity of Metal phase. Otherwise, the stream (1) to treat may contain S-impurities in a concentration below 10 wt ppm of S to limit the hydrogenation activity of metal phase as well as some N-compounds in a concentration below 10 wt ppm to neutralize acidity of the support.

In particular, when one element chosen from K, Na, P, B, S is present, its content is preferably less than or equal to 1% wt based on the weight of the catalyst, preferably less than or equal to 0.5% wt, most preferably less than or equal to 0.3% wt and advantageously less than or equal to 0.2% wt. The lower content in this element may be 200 wt ppm, preferably below 10 wt ppm, most preferably 50 wt ppm, more preferably 20 wt ppm.

For the two embodiments described above, secondary products may be formed in the reaction zone. For instance, dehydration reaction products may be formed. Such product may advantageously be separated and cracked in a dedicated olefin cracking reaction zone in order to improve the overall propylene yield, or may be recycled back to stream (1), optionally after rehydration.

For the two embodiments described above, butanol can be originating from a bio source compounds. In particular, butanol can be prepared via a selective fermentation of carbohydrate, reaction between ethanol and methanol on the basic catalysts or a reaction between CO and H₂.

For the two embodiments described above, the butanol could be extracted from a mixture of heavy alcohols synthesis from syn-gas.

The decarbonylation and decarboxylation reactions present the advantage of being selective reactions. Consequently, the propylene production process of the present invention presents the advantage of having a propylene yield of at least 70-73 wt % on carbon basis (based on iBuOH) via decarbonylation versus 55-60% on carbon basis while combining dehydration and cracking of the olefin produced.

It has been discovered that the (one-pot) direct decarbonylation of primary butanol is possible. Consequently, propylene can be directly prepared from primary butanol. But it can also be prepared from the corresponding aldehyde and/or carboxylic acid in the case of iso-butanol. Therefore conversion of iso-butanol into butyraldehyde and/or butyric acid does not need to be total. The remaining part of iso-butanol reaching the decarbonylation and/or decarboxylation section can be still converted into propylene. This is particularly advantageous because no complete intermediate purification is required in between the two reaction zone (B) and (C). When the same catalyst is used in both reaction zone (B) and (C), the operating conditions are adapted in each reaction zone to favor in each reaction zone the desired reaction.

### DETAILED DESCRIPTION OF THE INVENTION

As regards the butanol source (source of primary C4 alcohol), it is preferably prepared from a natural or bio source i.e. from a renewable carbon source (including but not limited to: carbohydrates, triglycerides, lignocellulosic biomass, agricultural wastes etc.). For instance, bio-butanol can be formed via transformation of natural carbohydrates. It can also be derived from lignocellulosic materials. Transformation routes include for instance fermentation by yeasts. As none limiting examples, butanol can be formed with process described in WO2008080124, WO2008143704, WO2011159894, WO2009140159, US2011313206, US2011195505, WO2012173659. Other preparation routes include for instance the conversion of synthesis gas (CO+H₂) that can be prepared from bio sourced materials, via gasification of lignocellulosic biomass. Example of the conversion of syn-gas into butanol is described in WO2012062338.

Iso-butanol could be also produced from other oxygenates, for example via the reaction between ethanol and methanol on basic catalysts.

The process of the present invention can treat normal butanol and iso-butanol, but other isomers such as 2-butanol or tert-butanol may be present in the stream (1). The preferred isomer of butanol is nevertheless iso-butanol.

In an embodiment, a single isomer of the butanol, for example iso-butanol, is used for transformation to propylene.

The stream (1) treated by the process of the present invention contains mainly primary C4 alcohol. It may contain all the butanol isomers in any proportion and preferably iso-butanol. It may also contain other compounds such as the oxidation product of the various butanol isomers, i.e. the corresponding aldehyde and carboxylic acid. In the case of iso-butanol, the oxidation compounds correspond to the derivated aldehyde and carboxylic acid of iso-butanol i.e. respectively iso-butyraldehyde and the isobutyric acid. The stream (1) may also contain small amounts of other alcohols from C3 to C6 and their corresponding oxidation product. Concentration of those other alcohols may vary from 1 ppm wt up to 5% wt for each. Those other alcohols may undergo similar transformation as the primary butanol and therefore form olefins from C2 to C5 that can be directly used or cracked into lower olefins.

As regards the expression “stream containing essentially primary C4 alcohol”, it shall be understood as a stream containing at least 50 wt % of primary C4 butanol, preferably at least 80 wt %, more preferably at least 90 wt % and the most preferably at least 95 wt % of the total weight content of alcohols i.e. without taking into account the optional presence of diluents or inert.

As regards the decarbonylation reaction, it consists in the selective removal of a CO molecule. In the case of the present invention, the co-product is an olefin containing one carbon atom less than the starting alcohol. Although it is not the predominant reaction, the alkanes corresponding to the olefins produced can be a by-product, i.e. propane when butanol is treated. Such by-product can advantageously be separated and treated in a dehydration process or in a steam cracker process in order to improve the overall propylene and olefins yield. In the case of iso-butanol dehydro-decarbonylation or iso-butyraldehyde decarbonylation, di-hydrogen (H₂) is formed in addition with the CO (see above reactions (1) and (2)).

Butanoic acid eventually formed is transformed to propylene via hydro-decarbonylation or decarboxylation (above reactions (3) and (4)). Butanoic acid may be co-fed to the reaction with butanol or butyraldehyde. Optionally, a neutralization of the butanoic acid stream to neutral pH is applied.

The mixture of CO and H₂ can advantageously be separated. It can be used as fuel gas. It can also advantageously be recycled to a butanol production unit if any. It can be converted in methanol as known in the art and further transformed into olefins via the Methanol To Olefin (MTO) reaction and/or transformed into a MTO unit wherein the heavy olefins formed are cracked into lower olefins in an Olefin Cracking Process.

As regards the decarboxylation reaction, it consists in the selective removal of a CO₂ molecule. It can either be performed on the protonated form of the carboxylic acid or on the corresponding soap. The decarboxylation leads either to an olefin or alkane containing one carbon atom less than the starting alcohol. When the alkane is produced, said alkane is separated and transformed in the corresponding olefins via dehydrogenation. Alkane can otherwise be separated and cracked in a steam cracker.

As regards the first embodiment of the invention, it consists in the direct transformation of primary butanol in a single reaction zone (A) to propylene. It is supposed to comprise the following one-pot transformations:
butanol to butyraldehyde via partial oxidation or dehydrogenation;
butyraldehyde to propylene, CO, H₂ via decarbonylation.

Without wishing to be bound by any theory, such direct transformation of butanol to propylene via decarbonylation may be possible due to the discovery that the catalyst for both reactions may be the same and both transformation occur under close reaction conditions.

Impurities contained in stream (1) such as alcohols can similarly be converted into olefins containing one carbon atom less than the starting alcohol and separated afterwards. The decarbonylation reaction may not be complete. Preferably at least 1 wt % of the primary butanol is converted, most preferably the conversion of primary butanol is of 10 wt % to 25 wt % and even more preferably the conversion of primary butanol is at least 80 wt % to 95 wt %. The unconverted compounds exiting the reaction zone (A) can be separated. In some case, part of the primary butanol can be dehydrated into butene that can be recovered at the exit of reaction zone (A) and then cracked into lower olefins in a dedicated olefin cracking unit. In some other cases, for instance, in the presence of trace of oxidizing agent such as O₂, part of primary butanol can be oxidized into the corresponding aldehyde and/or carboxylic acid.

Advantageously, such by-products can also be separated at the exit of the reaction zone (A) and recycled at the inlet of reaction zone (A) as they can further be converted in olefins. Alkanes may also be formed in reaction zone (A), they can advantageously be separated and dehydrogenated into the corresponding olefins or cracked in a steam cracker.

As regards the second embodiment of the invention, the stream (1) is first oxidized in a reaction zone (B) in order to convert at least part of the butanol into the corresponding aldehyde via partial oxidation and/or dehydrogenation, for example as follows:

Butanol→butyraldehyde+H₂

Butanol+½O₂→butyraldehyde+H₂O

The unconverted primary butanol can be separated at the exit of the reaction zone (B) and recycled at the inlet of the reaction zone (B). Once optionally purified, the butyraldehyde and butanol are sent to a second reaction zone (C) where it is converted into propylene, CO and H₂. The unconverted compounds can be separated and recycled either at the inlet of the reaction zone (C) and/or at the inlet of reaction zone (B). The mixture of CO and H₂ can advantageously be separated and transformed into butanol or other alcohols similarly as in the first embodiment.

The unconverted primary butanol exiting the reaction zone (B) may also not be separated and directly sent to the reaction zone (C) in order to be converted into the corresponding olefin. The second embodiment of the present invention presents therefore the advantage of being particularly versatile. Depending on the operating conditions and on the performances of reaction zone (B), the recycle of unconverted butanol can be adapted.

In the first reaction zone, butyraldehyde could be produced with selectivity higher than 90% below 450° C. Side reactions such as butanol or butyraldehyde overoxydation to CO₂, dehydration of the butanol to corresponding butenes, and formation of ethers may be enhanced at higher temperatures.

As regards the catalyst used, at least one non-acidic and optionally non-basic catalyst support of catalyst is used.

It has been discovered that the catalyst able to catalyze the decarbonylation reaction of butyraldehyde is also able to catalyze the reaction of decarbonylation of butanol as well as the oxidation of butanol to butyraldehyde. Consequently there is no need to have a pure alcohol or aldehyde stream to perform the decarbonylation reaction. Advantageously, the catalyst used to oxidize the alcohol into the corresponding aldehyde or carboxylic acid is the same as the catalyst used for the decarboxylation and/or decarbonylation reaction.

It has also been discovered that non-acidic and optionally non-basic catalyst support or catalyst can reduce the formation of by-products resulting from reaction of unconverted alcohol with butyraldehyde.

By “non-acidic catalyst support” or “non-acidic catalyst” is meant a catalyst or a support essentially deprived of Lewis and Brönsted acidic properties at least at the operating conditions of reaction.

By “non-basic catalyst support” or “non-acidic catalyst” is meant a catalyst or a support essentially deprived of Lewis and Brönsted basic properties at least at the operating conditions of reaction.

In other words, the catalyst used is essentially deprived of acidic sites, and optionally is also deprived of basic sites, and the support of the catalyst is also non-acidic and optionally non-basic, at least at the operating conditions of reaction.

In the present specification, the expression “based on the weight of the catalyst”:
refers to the weight of the carrier on which a metal phase is dispersed in the case of a supported catalyst,
or refers to the carrier alone when the catalyst does not comprise a metal phase dispersed on the carrier, for example when the catalyst is activated carbon,
or refer to the metal phase alone, when the metal phase is not supported.

Advantageously, the non-acidic and optionally non-basic catalyst support or catalyst may contain at most 2 wt % of Na based on the weight of the catalyst support or catalyst. For example, the Na content may range from 20 wt ppm to 2 wt %, preferably from 200 wt ppm to 2 wt %.

In combination or alternatively, the non-acidic and optionally non-basic catalyst support or catalyst may contain at most 2 wt % of S based on the weight of the catalyst support or catalyst. For example, the S content may range from 1 wt ppm to 2 wt %.

Such low content of Na and/or S may limit side reactions leading to heavies.

In any embodiment, the catalyst may comprise an active transition metal, preferably dispersed on a carrier. The active transition metal may be chosen in the groups IB, IIB, IVB, VB, VIB, VIIIB. The metals can be in the metallic state, in an oxidized state, in a partially reduced oxide state.

Also metals of Groups IIIA and IVA are known to be active.

In case of first embodiment, the decarbonylation function may be combined with the oxidative one. The active metal compounds may be chosen among Groups VB, VIB, and VIIIB metals. They can be in the metallic state, in an oxidized state, in a partially reduced oxide state. Also Group IB metals are known to be active.

The most preferred metals are Fe, Mo—, Pd, Ag, Cu, Ni, Cr. The metal phase could be supported on non-acid materials selected from passivated alumina, activated carbon, amorphous alumina phosphates, silica, ceria, zirconia, titania, clays etc.

Advantageously, in order to promote decarbonylation and limit the hydrogenation of propylene to propane, a second metal component may be present. So, activity and selectivity of catalysts used in the first reaction zone of the second embodiment can still further be improved by employing bimetallic compounds. Metal may be chosen from groups VIB and VIIIB. Typical examples are CoMo, FeMo, NiW and NiMo catalysts used in the first reaction zone.

Advantageously, a typical dehydrogenation/oxidation catalyst that may be used in the first reaction zone of the second embodiment may contain at least one metal chosen from groups IB, IIB, IVB, VB, VIB, VIIIB, IIIA, lanthanides. By way of example, the catalyst may be based on Ga-, Zn-, Pt-, Pd-, Cr-, Ni-, Co-, Fe-, Cu-, Ag-, Mo-, Ce-, Zr-, Ru-, Rh-, Ti, Ta, Nb, Os, Ir-containing materials. The catalyst may contain at least two metals chosen among the above groups, preferably among groups IB, IVB, VB, VIIIB, IIIA, IVA. A bimetallic catalyst may be selected from: Cu—Pd, Fe—Mo, Cu—Ni, Cu—Co, Cu—Pt, Fe—Pd, Co—Pd, Ni—Pd, Pt—Pd, Ag—Pd, Au—Pd, Fe—Pt, Ni—Pt, Pt—Sn, Pt—Pb, Pd—Sn, Pd—Pb, Au—Pd, Nb—Pd, Ga—Pd, Zr—Pd and many others.

Advantageously, catalysts that may be used in the second reaction zone of the second embodiment for selective decarbonylation may contain at least two metals chosen in the groups IB, IVB, VB, VIB, VIIIB, IIIA, IVA. Examples of couple of metals that may be present are: Cu—Pd, Cu—Ni, Cu—Co, Cu—Pt, Fe—Pd, Co—Pd, Fe—Mo, Ni—Pd, Pt—Pd, Ag—Pd, Au—Pd, Fe—Pt, Ni—Pt, Pt—Sn, Pt—Pb, Pd—Sn, Pd—Pb, Au—Pd, Nb—Pd, Ga—Pd, Zr—Pd and many others. The preferred catalysts are based on bimetallic catalysts containing Pd.

The metal phase of the catalyst may be supported by a carrier or not. in the present specifications the terms “support” and “carrier” are indifferently used to design a compound on which the metal phase is dispersed.

The concentration of metal on the carrier may be at least of 0.01 wt %, for example from 0.05 wt % to 15 wt %, preferably from 0.2 to 3 wt %, most preferably from 0.3 wt % to 10 wt %, based on the weight of the catalyst.

The activity and selectivity is also influenced by the characteristics of the carrier for the metal compound. The carrier can influence the dispersion of the metal or metal compound, the particle size of the metal or metal compound and the electronic properties of the metal or metal compound.

Suitable carriers may be selected from passivated alumina, in particular passivated (non-acid) alumina, activated carbon, hydrotalcites, metal silicates, perovskytes, silica-magnesia, amorphous alumina phosphates, AlPO4's, phalocianides, silica, ceria, zirconia, titania, clays and their mixtures.

The preferred metal silicate is a calcium silicate with a very open and accessible pore structure. An even more preferred metal silicate comprises a synthetic crystalline hydrated calcium silicate having a chemical composition of Ca₆Si₅O₁₇(OH)₂ which corresponds to the known mineral xonotlite (having a molecular formula 6CaO.6SiO₂.H₂O).

Generally, a synthetic hydrated calcium silicate is synthesized hydrothermally under autogeneous pressure. A particularly preferred synthetic hydrated calcium silicate is available in commerce from the company Promat of Ratingen in Germany under the trade name Promaxon. Other examples of metal silicates comprising alkaline earth metals include CaAl₂Si₂O₈, Ca₂Al₂SiO₇, CaMg(Si₂O₆)ₓ, as well as mixtures thereof.

Silica, activated carbon or passivated alumina are particularly preferred.

In order to suppress the dehydration and condensation by-products, the carrier may preferably not have any acid properties and any basic properties, at least under the operating conditions of the reaction.

The acidity of the carrier may be passivated by K, Na addition to the concentration of 2 wt % at most of the metal based on the weight of the catalyst or by a selecting a basic material. Known carriers include activated carbon, passivated (non-acid) alumina, silica, titania, zirconia, amorphous alumina phosphates, ALPO4's, ceria, clays, Silica-Based Crystalline Organic-Inorganic Hybrid Materials (for example Eni Carbon Silicate (ECS)), different modifications of SiC.

The acid-base properties of the carrier can be very important for several reasons.

Often, the above kinds of catalysts are supported on alumina-type carriers which have been modified by the addition of one or more alkali metal or alkaline earth metal compounds, which tend to moderate the acidity of the alumina in the carrier and hence increase the selectivity and the potential lifetime of the catalyst.

The catalyst used may also be activated carbon.

Advantageously, the non-acidic and optionally non-basic catalyst support or catalyst may comprise, or consist of, an activated carbon. Activated carbon in its broadest meaning includes a wide range of amorphous carbon-based materials characterized by a highly developed porosity and an extended interparticulate surface area. They are generally prepared in two steps: carbonization of the carbonaceous raw material at temperatures below 1073 K in an inert atmosphere followed by activation of the carbonized product. Thus, all carbon-rich materials can be converted into activated carbon, although the characteristics of the final solid will be different, depending on the nature of the raw materials used, the nature of activating agent, and the conditions of the carbonization and activation processes.

Porous carbon materials are the preferred activated carbons: (i) carbon foams with desired architecture of pores for structural and thermal applications, used as templates for making ceramics, (ii) activated carbons consisting of porous carbons with added active surface chemical groups.

a. Powdered Activated Carbon (PAC)

Usually, active carbons are made as powders or fine granules that not exceed 1.0 mm in size and with an average diameter between 15 and 25 μm. Thereby, they present a large internal surface with a small diffusion distance.

b. Granulated Activated Carbon (GAC)

Granulated activated carbons have a relatively larger size particles than PAC. They therefore have a smaller external surface.

c. Extruded Activated Carbon (EAC)

Extruded activated carbons (pressed pellets) are usually made by mixing PAC with a suitable binder to make a paste which is extruded under high pressure into a cylindrical shape with diameters from 0.8 to 10 mm.

d. Activated Carbon Cloth (ACC)

Activated carbon cloths with very high surface area (up to 2000 m²/g) have been the latest addition in the family of activated porous carbons. They can be obtained by carbonization and activation of previously impregnated organic polymers, mostly of cellulose origin.

Activation process is used to increase pores sizes of the carbon support which are developed during the carbonization and to create some new porosity thus resulting in the formation of a well-developed and readily accessible pore structure with very large surface area. Activation can occur in one of two ways: physical or chemical treatment.

### Physical Activation

It is a gas treatment by which the porous structure and surface area of the carbonized product will be developed. This treatment is carried out in the temperature range of 1073-1273 K in presence of suitable oxidizing gases such as steam, CO₂, or a combination of both.

### Chemical Activation

In chemical treatment, the process is slightly different from the physical activation of carbon. First, carbonization and activation occur simultaneously in this case. A bath of acid, base or other chemicals is prepared and the material impregnated in it. The bath is then heated to temperatures of 773-1173 K for 0-3 h, much less than the heat needed for gas activation. The carbonaceous material is carbonized and then activated, all at much quicker pace than gas activation. However, some heating processes cause trace elements from the bath to adsorb to the carbon, which can result in impure or ineffective active carbon. For that, washing post-treatments is therefore necessary to remove these impurities. The most common chemical agents used for chemical activation are ZnCl₂, KOH, H₃PO₄ and less frequently K₂CO₃.

A suitable catalyst may be produced by impregnation of a metal phase on the support. Preferably an incipient wetness impregnation technique is employed where the pores of the support are filled with a volume of solution containing the metal. In this technique, the dried catalyst is impregnated with a solution of a salt of the at least one Group, IB, IIB, VIB, VIIB or VIIIB metal, for example a halide of the metal, in particular the Group VIIIB metal chloride. The amount of the metal salt is calculated to provide a desired metal content on the support, for example a metal content of from 0.01 to 15 wt % based on the weight of the supported catalyst, most preferably from 0.2 -3 wt % based on the weight of the supported catalyst. The impregnated solid is dried first under vacuum and subsequently at elevated temperature.

Finally, the product is calcined, for example at a temperature of about 500° C. for a period of about 3 hours.

Alternatively an excess of solution is used during the impregnation step and the solvent is removed by evaporation. Depending on the properties of the impregnation solution and the carrier, the active metal phase can have different locations: (1) the metal or metal compound is concentrated in a thin layer close to the external surface, this may be referred to as an “egg-shell mode”, (2) the metal or metal compound is concentrated in a thin layer below the surface, but not penetrating to the center, this may be referred to as an “egg-white mode”, (3) the metal or metal compound is concentrated in a small zone near the centre of the particle carrier, this may be referred to as an “egg-yolk mode”, and (4) the metal or metal compound is uniformly distributed throughout the particle carrier. The way that the metal precursor will interact with the carrier depends on the isoelectric point (IEP) which is the pH at which the particle of the carrier in an aqueous solution has no net charge. At pH's above the IEP, cations will be adsorbed, because the surface carries a negative charge; below the IEP, only anions will be adsorbed, because the surface carries a positive charge. During the contact of the impregnating solution and the carrier, ion exchange can also occur. The impregnating solution may be altered by adding complexing agents, which can change the charge of the metal precursor. In another technique, competing ions may be added to improve the spreading of the metal precursor over the carrier.

The present invention is however not limited by a method of preparation, provided the catalyst support or catalyst presents the non-acidic and optionally non-basic properties required for the present invention.

Before use, the prepared catalyst may optionally be activated in oxidizing atmosphere with a very low heat rate, preferable below 5° C./min, more preferably about 0.5° C./min at a temperature up to 600° C., preferably up to 500° C.

Before use in the process of the present invention, eventually after the above optional oxidation activation treatment, a reduction in presence of di-hydrogen (H₂) may be performed on the catalyst at a temperature of at least 400° C. for at least 1 hour. The reduction may be performed at a temperature from 400° C. to 600° C., preferably from 400° C. to 500° C. The duration of the reduction may range from 1 to 48 hours. To perform this activation by reduction, the catalyst may be contacted with a pure H₂ flow, or a flow containing at least 10 mol % of H₂.

As regards the measurement of the catalyst support basic sites or catalyst basic sites it can either be defined as the ability to accept protons as defined by Brönsted, or as a substance, which has an unshared electron pair with which it can form a covalent bond with an atom, molecule or ion as defined by Lewis.

In particular, the catalyst support or catalyst useful herein is classified as non-basic if it has an uptake of carbon dioxide of less than 100 μmol/g of the catalyst support or catalyst, preferably less than 90 μmol/g of the catalyst support or catalyst. Typically, the catalyst support or catalyst useful herein has a carbon dioxide uptake of 1 to 80 μmol/g preferably of 1 to 50 of the catalyst support or catalyst. The measurement of the catalyst basic sites (TPD CO2) should preferably be performed before any optional impregnation of metal(s) preferably on the catalyst support.

In order to determine the carbon dioxide uptake of a catalyst support or catalyst, the following special procedure is adopted (see detail description of the possible procedure in examples). A sample of the catalyst support or catalyst is dehydrated by heating the sample from room temperature to 550° C. in flowing helium until a constant weight, the “dry weight”, is obtained. The temperature of the sample is then reduced to 40′ C. and carbon dioxide is passed continuously over the sample. After removing the physisorbed CO₂ in He flow, the analysis, the thermo desorption analysis is started. The amount of CO₂ desorbed in the temperature interval between 40° C. and 500° C. is taken into consideration. The measurement of the catalyst support acidic sites or catalyst acidic sites (TPD NH3) should preferably be performed before any optional impregnation of metal(s) preferably on the catalyst support.

As regards the measurement of the catalyst support acid sites or catalyst acid sites it can either be defined as the ability to give protons as defined by Brönsted, or as the ability to accept an electron pair as defined by Lewis. The measurement of the acidity is performed via temperature-programmed desorption of ammonia (see detail description of the possible procedure in examples).

In particular, a catalyst support or catalyst useful herein is classified as “non-acid” according to invention if it has an uptake of ammonia of less than 150 μmol/g of the catalyst, preferably less than 100 μmol/g of the catalyst most preferably of less than 50 μmol/g. Typically, the catalyst support or catalyst useful herein has an ammonia uptake of 0.1 to 80 μmol/g of the catalyst support or catalyst.

As regards the optional diluent used for the two embodiments, it may be injected in stream (1), separated after each reaction zone and recycled.

It can also be simply injected in stream (1) and separated after all the reactions are performed. Any diluent which is able to play a thermal flywheel role and which is inert for the reaction may be used. Suitable diluent are for example water vapor, N₂, Ar, any other inert component for the reaction and their mixtures.

For the two embodiments described above, when a diluent is added, it can advantageously be added at a maximum concentration of 80 wt % based on the mixture of diluent+stream (1). The dilution is advantageous because the reaction occurs with volume expansion and allows maintaining low partial pressure of the reagent at higher overall process pressure. In addition, the dilution limits secondary reaction of butyraldehyde and formation of propane by hydrogenation of propylene, So, the selectivity could be improved.

For the two embodiments described above, a presence of the basic compounds in the diluent is advantageous. Ammonia, nitriles or some other basic compound stable at the reaction temperature is desirable. A solution of potassium carbonate could also be used. These compounds could be used in co-injection with a normal diluent. The role is to passivate the residual acidity of the catalyst support in order to avoid dehydration activity.

As regards the operating conditions of the reactions zone of the two embodiments, the temperature preferably ranges from 150° C. to 500° C. preferably from 200° C. to 450° C. and most preferably from 250° C. to 400° C. The pressure ranges from 0.01 MPa (1 bar) to 10 MPa (100 bars) preferably from 0.1 MPa (1 bar) to 5 MPa (50 bars), most preferably from 2.5 MPa (25 bars) to 3.5 MPa (35 bars). The gas hourly space velocity (GHSV) may range from 0.5 h⁻¹ to 20 h⁻¹, most preferably from 1 h⁻¹ to 5 h⁻¹.

In the second embodiment, the reaction conditions in the first and the second reaction zones could be the same or different.

Advantageously, the reaction temperature in the first zone of the second embodiment is below 450° C. preferably below 400° C., more preferably below 390° C. The temperature may range from 150° C. to 450° C., preferably ranging from 250° C. to 390° C.

The partial pressure of butanols may range from 0.01 MPa ((0.1 bar) to 2.5 MPa (25 bars), advantageously from 0.05 MPa (0.5 bar) to 15 MPa (15 bars).

Advantageously, the reaction temperature in the second reaction zone of the second embodiment is higher than the reaction temperature in the first reaction zone. Second reaction zone (C) may be operated at a temperature ranging from 150° C. to 500° C., preferably ranging from 250° C. to 450° C.

The partial pressure of butyraldehyde may range from 0.01 MPa (0.1 bar) to 1 MPa (10 bars), advantageously from 0.05 MPa (0.5 bar) to 0.8 MPa (8 bars). The second reaction zone is preferably free of oxidative medium (oxygen, air, NO etc) and advantageously contains less than 10 mol % of butanols.

As regards the reaction zones in the two embodiments, each reaction zone may comprise one or several reactors. Reactors can be a fixed bed reactor, a moving bed reactor or a fluidized bed reactor. A typical fluid bed reactor is one of the FCC type used for fluidized-bed catalytic cracking in the oil refinery. A typical moving bed reactor is of the continuous catalytic reforming or dehydrogenation type. The reaction may be performed continuously in a fixed bed reactor configuration using a pair of parallel “swing” reactors. The various preferred catalysts of the present invention have been found to exhibit high stability. This enables the process to be performed continuously in two parallel “swing” reactors wherein when one reactor is operating; the other reactor is undergoing catalyst regeneration. The catalyst of the present invention also can be regenerated several times. In case of the two reaction zones, the process could be operated with one or two different reactor types. In preferred embodiment, the “swig” fixed reactors system is used.

As regards the propylene produced, it can be further purified into polymer grade propylene. It can be also used as such for the propylene conversion processes, which allow higher propane concentration like propylene epoxidation or oligomerization. It can also be polymerized and used to prepare any objects known in the art. It can also be further transformed into other valuable molecules.

As regards the CO+H₂ produced, and eventually the CO2 produced, they can advantageously be separated.

The mixture CO+H₂ may be used as fuel gas and burned out to produce energy or may be subject to a contact with an appropriate catalyst to make hydrocarbons or oxygenates via the conventional syn-gas conversion routes. The syn-gas conversion routes are known per se and may include Fischer-Tropsch, olefins synthesis, aromatics or oxygenates as C1-C5 alcohols (methanol, ethanol etc).

The mixture CO+H₂ can advantageously be recycled to a selective butanol preparation unit or used to produce alcohol from C1 to C5, preferably methanol, or any combination of thereof. In particular, the mixture CO+H₂ could be co-injected into heavy alcohols synthesis reactor with recycled light alcohols to produce more butanols.

The reaction gaseous products rich in CO and/or CO₂/H₂ could also be converted to liquid products by supported or unsupported microorganism in bio-reactor, i.e. via microbial fermentation. The preferred liquid products are methanol, acetaldehyde, formic acid, formaldehyde, ethanol, acetone, isoprapanol, acetic acid, propionic acid, ethylacetate, butanols, butyraldehyde, butanoic acid etc.

The CO and di-hydrogen mixture could be also used for a selective transformation of the ethylene to propanal, acetone, and (n-, i-) propanols. The oxygenates could be further transformed to the propylene via the well know processes.

The CO and di-hydrogen mixture could be also co-reacted with ethylene to hydro oligomerise ethylene on the Co-, Ni-containing catalyst to heavy hydrocarbons (Edius reaction).

### DESCRIPTION OF THE FIGURES

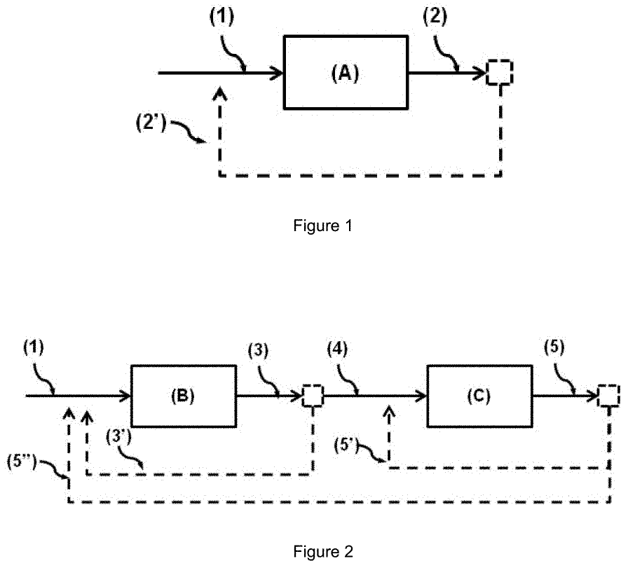

The FIG. 1 represents a simplified process scheme of the first embodiment of the present invention. A stream (1) comprising essentially primary butanol is sent to a reaction zone (A). In this reaction zone, primary butanol is decarbonylated in order to produce propylene, carbon monoxide and di-hydrogen. Unconverted primary butanol can optionally be separated and recycled back to stream (1) via stream (2′) (dashed line). Optionally, the diluent present in stream (1) may also be separated at the exit of reaction zone (A) and recycled in stream (1) via stream (2).

The FIG. 2 represents a simplified process scheme of the second embodiment of the present invention. A stream (1) comprising essentially primary butanol is sent to a reaction zone (B) where it is at least partially converted via oxidation into the corresponding aldehyde. The stream (3) exiting the reaction zone (B) contains unconverted primary butanol, the corresponding aldehyde and maybe also the corresponding carboxylic acid in case the oxidation reaction was not completely selective. Optionally part or all of the unconverted primary butanol and/or the diluents can be separated and recycled into stream (1) via stream (3′) (dashed line) to obtain a purified stream (4). The stream (4) is then sent to a reaction zone (C) where the decarbonylation and optionally the decarboxylation reaction are performed. At the exit of the reaction zone (C), the stream (5) containing mainly propylene, CO and H₂ is obtained. This stream (5) can optionally be purified to separate propylene from the CO and H₂ that can further be recycled into a butanol or methanol production unit (not presented on the figure). The stream (5) can optionally be purified to separated unconverted compounds and diluents and recycle them either at the inlet of reaction zone (C) through stream (5′) (dashed line) or at the inlet of reaction zone (B) through stream (5′) (dashed line).

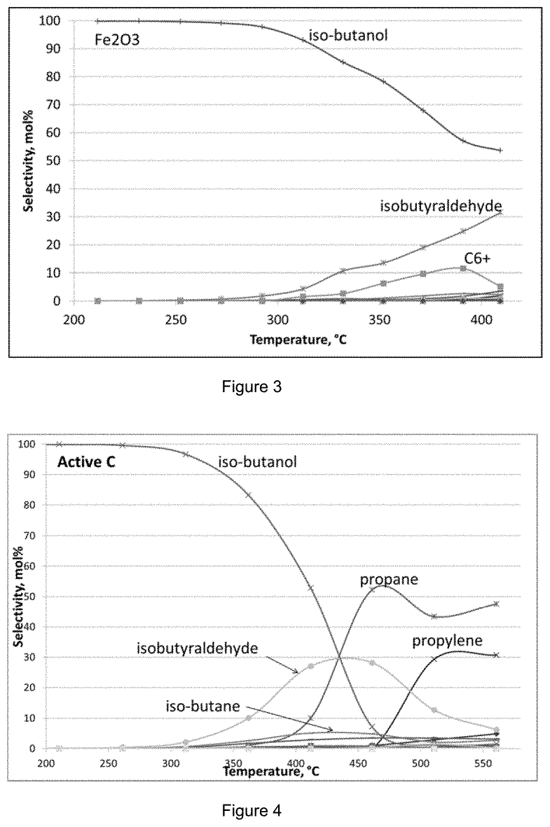

The FIG. 3 represents the selectivity of the products formed as a function of the temperature in the iso-butyraldehyde formation from iso-butanol (example 12).

The FIG. 4 represents the selectivity of the products formed as a function of the temperature in the iso-butyraldehyde and propylene formation from iso-butanol (example 13).

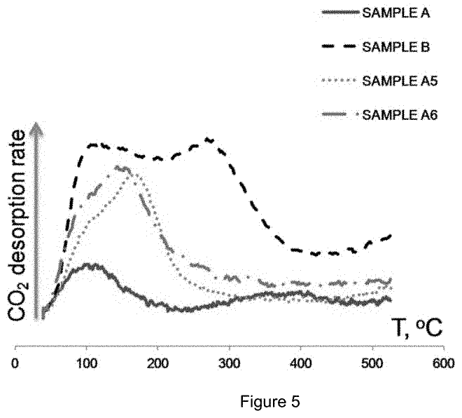

The FIG. 5 represents the CO₂ desorption curves of the samples normalized on the weight (see procedure for the CO₂ uptake measurements).

### PROCEDURE FOR BASICITY MEASUREMENT (TPD CO2 or CO₂ Uptake)

The measurement of Temperature-programmed desorption of CO₂ is performed in a Pyrex®™ cell containing about 0.4 g of sample in form of the fraction 35-45 mesh. The cell is placed in an oven of the AUTOCHEM II 2920 equipped with TCD detector and the following steps are carried out:

Activation: this step is performed under a flow rate of dried (over molecular sieve e.g. 3 A or 4 A) He of 50 cm³/min (<0.001% of water). The temperature is increased from room temperature to 550° C. with a rate of 10° C./min. The temperature is then maintained at 550° C. during 1 h. The temperature is then decreased to 40° C. with a rate of 10° C./min.

Saturation: this step is performed at 40° C. During two hours, the solid is put in contact with a flow of 30 cm³/min of a dried (over molecular sieve e.g. 3 A or 4 A, <0.001% of water) pure CO₂. Then, during the next 1 h, the solid is put in contact with a flow rate of 50 cm³//min of dried (over molecular sieve e.g. 3 A or 4 A, <0.001% of water) He to remove the physisorbed CO₂.

Analysis: this step is performed under a flow of 50 cm³/min of dried (over molecular sieve e.g. 3 A or 4 A, <0.001% of water) He. The temperature is increased to 500° C. with a rate of 10° C./min. Once the temperature of 500° C. has been reached, it is maintained for 1 h. The cell is then cooled down and weighted. The amount of CO₂ desorbed in the temperature range from 40° C. to 500° C. from the solid is referenced to the weight of the sample.

### PROCEDURE FOR ACIDITY MEASUREMENT (TPD NH₃)

The measurement of Temperature-programmed desorption of ammonia is performed in a Pyrex®™ cell containing about 0.4 g of sample in form of the fraction 35-45 mesh. The cell is placed in an oven of the AUTOCHEM II 2920 equipped with TCD detector and the following steps are carried out:

Activation: this step is performed under a flow rate of dried (over molecular sieve e.g. 3 A or 4 A) He of 50 cm³/min (<0.001% of water). The temperature is increased from room temperature to 600° C. with a rate of 10° C./min. The temperature is then maintained at 600° C. during 1 h. The temperature is then decreased to 100° C. with a rate of 10° C./min.

Saturation: this step is performed at 100° C. During a first hour, the solid is put in contact with a flow of 30 cm³/min of a dried (over molecular sieve e.g. 3 A or 4 A, <0.001% of water) mixture of 10 weight % of NH₃ diluted in He. Then, during the next 2 h, the solid is put in contact with a flow rate of 50 cm³ /min of dried (over molecular sieve e.g. 3 A or 4 A, <0.001% of water) He to remove the physisorbed NH₃.

Analysis: this step is performed under a flow of 50 cm³/min of dried (over molecular sieve e.g. 3 A or 4 A, <0.001% of water) He. The temperature is increased to 600° C. with a rate of 10° C./min. Once the temperature of 600° C. has been reached, it is maintained for 1 hr. The cell is then cooled down and weighted. The amount of NH₃ desorbed from the solid is referenced to the weight of the sample.

### EXAMPLES

The iso-butanol conversion is the ratio (iso-butanol introduced in the reactor−iso-butanol leaving the reactor)/(iso-butanol introduced in the reactor).

The iso-butanal conversion is the ratio (iso-butanal introduced in the reactor−iso-butanol leaving the reactor)/(iso-butanol introduced in the reactor),

The propylene selectivity is the ratio, on carbon basis, (propylene leaving the reactor)/(iso-butanol or iso-butanal converted in the reactor).

The selectivity in C4 olefins is the ratio, on carbon basis, (C4 olefins leaving the reactor)/(iso-butanol or iso-butanol converted in the reactor).

The isobutanal selectivity is the ratio, on carbon basis, (isobutanal leaving the reactor)/(iso-butanol converted in the reactor),

The C3's cut purity is the ratio, on carbon basis, (propylene leaving the reactor)/(propylene+propane leaving the reactor). It means the propylene purity is the percentage of propylene, on a carbon basis, present in the C₃ cut, containing close-boiling compounds, recovered in the stream leaving the reactor.

The following notations were used:

C3=:propylene,

C4=:C4 olefin,

i-BuOH:iso-butanol,

i-butanal:iso-butanal (iso-butyraldehyde).

### Example 1

A catalyst composition was produced by extrusion of 50 g of Nyasil 20 (Nyacol—an amorphous silica powder) with 45 g of silica sao Ludox HS-40 (SiO₂ 40 wt %, W. Grace). The obtained extruded body was dried for 24 h at room temperature, dried for 24 h at 110° C., and calcined at 500° C. for 6 h in static air with a heating rate of 1° C./min.

The sample is hereinafter identified as A (TPD NH_{3—} 30 μmol/g, TPD CO_{2—} 42 μmol/g).

### Example 2 (Comparative)

50 g of MgCO₃ (Aldrich) was calcined at 600° C. for 10 h (1° C./min heating rate) to produce MgO. The 21 g of the synthesized MgO was extruded with 22.5 g of silica sol Ludox HS-40 (SiO₂ 40 wt %, W. Grace). The obtained extruded body was dried for 24 h at room temperature, dried for 24 h at 110° C. and calcined at 500° C. for 6 h in static air with a heating rate of 1° C./min.

The sample is hereinafter identified as B (TPD NH_{3—} 75 μmol/g TPD CO_{2—} 383 μmol/g).

### Example 3

20 g of the sample A (TPD NH_{3—} 30 μmol/g, TPD CO_{2—} 42 μmol/g) was incipient wetness impregnated with 1.005 g of Pd(NO)₃×2H₂O to introduce 2 wt % of Pd to the sample. The impregnated sample was maturated during 2 h at room temperature, dried for 24 h at 110° C. and calcined at 500° C. for 6 h in static air with a heating rate of 1° C./min.

The sample is hereinafter identified as A1.

### Example 4

20 g of the sample A (TPD NH_{3—} 30 μmol/g, TPD CO_{2—} 42 μmol/g) was incipient wetness impregnated with a solution containing 0.5 g AgNO₃ and 8.88 g Ni(NO₃)₂×6H₂O to introduce 0.5 wt % of Ag and 6 wt % Ni over the sample. The impregnated sample was maturated during 2 h at room temperature, dried for 24 h at 110° C., and calcined at 500° C. for 6 h in static air with a heating rate of 1° /min.

The sample is hereinafter identified as A2.

### Example 5

30 g of the sample A (TPD NH_{3—} 30 μmol/g, TPD CO_{2—} 42 μmol/g) was incipient wetness impregnated with 0.15 g of Pd(NO)₃×2H₂O to introduce 0.2 wt % of Pd to the sample. The impregnated sample was maturated during 2 h at room temperature, dried for 24 h at 110° C., and calcined at 500° C. for 6 h in static air with a heating rate of 1° C./min.

The sample is hereinafter identified as A3.

### Example 6

30 g of the sample A (TPD NH_{3—} 30 μmol/g, TPD CO_{2—} 42 μmol/g) was incipient wetness impregnated with a solution containing 0.5 g AgNO₃ and 0.15 g of Pd(NO)₃×2H₂O to introduce 1 wt % of Ag and 0.2 wt % of Pd over the sample. The impregnated sample was maturated during 2 h at room temperature, dried for 24 h at 110° C., and calcined at 500° C. for 6 h in static air with a heating rate of 1° C./min.

The sample is hereinafter identified as A4.

### Example 7 (Comparative)

20 g of the sample B (TPD NH_{3—} 75 μmol/g, TPD CO_{2—} 383 μmol/g) was incipient wetness impregnated with a solution containing 0.5 g AgNO₃ and 0.15 g of Pd(NO)₃×2H₂O to introduce 1 wt % of Ag and 0.2 wt % of Pd over the sample. The impregnated sample was maturated during 2 h at room temperature, dried for 24 h at 110° C., and calcined at 500° C. for 6 h in static air.

The sample is hereinafter identified as B1.

### Example 8 (Comparative)

5 g of the sample A1 (2 wt % Pd/SiO₂) was incipient wetness impregnated with 0.1 g Na₂S (0.7 ml H₂O/1 g of solid, Pd/S atomic ratio ˜0.74, Na/Pd −2.7). The impregnated sample was maturated during 2 h at room temperature, dried for 16 h at 120° C.

The sample is hereinafter identified as A5 (TPD NH_{3—} 42 μmol/g, TPD CO_{2—} 146 μmol/g).

### Example 9 (Comparative)

15 g of the A1 (2 wt % Pd/SiO₂) was incipient wetness impregnated with 2.2 g of Pd(NO)₃×2H₂O to produce a sample containing 5 wt % of Pd to the sample (0.8 ml H₂O/1 g solid). The impregnated sample was maturated during 2 h at morn temperature, dried for 24 h at 110° C., and calcined at 500° C. for 6 h in static air with a heating rate of 1° C./min.

Then, the calcined sample was reduced in H₂ flow (10 Nl/h) for 3 h at 450° C.

The total amount of the produced sample after reduction with hydrogen was incipient wetness impregnated with 0.3 g Na₂S+0.1 g NaOH (0.7 ml H₂O/1 g solid, Pd/S atomic ratio ˜2.92, Na/Pd ˜0.85, 2.8 wt % S). The impregnated sample was maturated during 2 h at room temperature, dried for 16 h at 120° C.

The sample is hereinafter identified as A6 (TPD NH_{3—} 25 μmol/g, TPD CO_{2—} 143 μmol/g).

### Example 10

Catalytic test was performed in a down flow stainless-steel reactor tube with an internal diameter of 11 mm. 4 g of crushed catalyst (35-45 mesh) was loaded. The reactor temperature was increased at a rate of 60° C./h to 450° C. under nitrogen flow 10 Nl/h. Then the catalyst was treated for 1 hour at 450° C. in nitrogen followed by reduction in di-hydrogen flow 10 Nl/min for 2 h at atmospheric pressure. Afterwards the reactor was purged with nitrogen followed by cooling down to the reaction temperature in nitrogen flow and pressurization if it was required with nitrogen.

Analysis of the products is performed by using an on-line gas chromatography.

### Example 11

Catalyst test according to the example 10 was performed with a pure iso-butanol as feed on catalysts A1, B1, A6.

The conditions and results are gathered in table 1.

TABLE 1

| | T, ° C. | | |
|---|---|---|---|
| | 375 | 375 | 375 |
| | A1 | B1 | A6 |
| | invention | comparative | comparative |
| WHSV, h⁻¹ | 1 | 1 | 1.6 |
| Pressure, barg | 5 | 5 | 0.5 |
| Conversion of i-BuOH, % | 15.4 | 9.2 | 6.3 |

Selectivity on C-basis, %

| | | | |
|---|---|---|---|
| Decarboxylation (C3=) | 2.3 | 2.9 | 1.6 |
| Dehydration (C4=) | 8.8 | 20.2 | 4.8 |
| Iso-butanal | 79.2 | 53.6 | 58.7 |
| heavies | 9.7 | 23.3 | 34.9 |

The example demonstrates a possibility to produce iso-butyraldehyde and propylene directly from iso-butanol. However, basic support (MgO— catalyst B1) or post-synthetic modification with basic compounds (Na— catalyst A6) leads to significantly higher amount of heavies due to side reactions. This shows that the use of non-basic type of catalyst permits reducing the amount of heavies, which are supposed to be formed via side reaction between butyraldehyde and non-reacted butanol in direct transformation or at the step of butanol conversion to butyraldehyde in the first reaction zone of a two stage process.

### Example 12 (Iso Butyraldehyde Formation from Isobutanol)

Catalytic test was performed in a quartz reactor of 5 mm internal diameter. Before the test, catalyst has been activated in situ for 1 h at 500° C. (2° C./min) in flow of He (50 ml/min). Isobutanol of 99% purity from Sigma-Aldrich has been used (0.806 g/ml density @15° C.; 0.085 wt % H₂O; 8 ppm S). The feed was sent to the reactor via a thermostatic saturator containing isobutanal at 75° C. at a pressure close to atmospheric (1.1 bar). The test was performed on 0.5 g of crushed Fe₂O₃ catalyst (>99%, Sigma Aldrich, 35-45 mesh), at weigh hour space velocity 9.4 h⁻¹ in a temperature range from 100 to 550° C.

FIG. 3 shows the evolution of selectivity of the products formed as a function of the temperature.

The example demonstrates feasibility of iso-butyraldehyde production from iso-butanol.

### Example 13 (Iso-butyraldehyde and Propylene Formation from Iso-butanol)

Catalytic test was performed in a quartz reactor of 5 mm internal diameter. Before the test, catalyst has been activated in situ for 1 h at 500° C. (2° C./min) in flow of He (50 ml/min). Iso-butanol of 99% purity from Sigma-Aldrich has been used (0.806 g/ml density @15° C.; 0.085 wt % H₂O; 8 ppm S). The feed was sent to the reactor via a thermostatic saturator containing iso-butanol at 75° C. at a pressure close to atmospheric (1.1 bar). The test was performed on 0.5 g of activated carbon catalyst (granulated, Sigma Aldrich, 35-45 mesh), at weigh hour space velocity 9.4 h⁻¹ in a temperature range from 100 to 550° C.

FIG. 4 shows the evolution of selectivity of the products formed as a function of the temperature.

The example shows a possibility to produce iso-butyraldehyde and propylene directly from iso-butanol. It's evident that the iso-byraldehyde is a true intermediate product in the reaction.

### Example 14

Catalyst test according to the example 10 was performed with a pure iso-butanal as feed on the catalysts A1 (300° C., atmospheric pressure, WHSV-1 h⁻¹) and A2 (300° C., 5 bars, WHSV-1 h⁻¹).

The results are gathered in table 2.

The data illustrate that both catalysts may be used to transform iso-butanal to propylene. However, Ni-containing catalyst (A2) is slightly less efficient. In order to reach the same conversion level, more severe conditions are required.

TABLE 2

| Catalyst | A1 | A2 |
|---|---|---|
| Conversion, % | 54 | 50 |

Selectivity on C-basis, %

| | | |
|---|---|---|
| Decarbonylation (C3H6 + C3H8) | 96 | 78 |
| Hydrogenation (Iso-butanol) | 1 | 14 |
| Oxidation (butanoic acid) | 2 | 2 |
| Heavy products (condensation) | 2 | 6 |

### Example 15

Catalyst test according to the example 10 was performed with a pure iso-butanal as feed on the catalysts A1 under different operating conditions C1, C2, C3, C4. Results are given in table 3. The different conditions and results are gathered in table 3. All the tests have been performed for 1 week. It was observed stable results for all the duration of the tests.

TABLE 3

| Operating conditions | C1 | C2 | C3 | C4 |
|---|---|---|---|---|
| WHSV, h⁻¹ | 1 | 1 | 2 | 4 |
| T, ° C. | 250 | 300 | 300 | 300 |
| P, barg | 0.5 | 0.5 | 0.5 | 0.5 |
| H₂/CO | 0.67 | 0.63 | 0.66 | 0.7 |
| C3 =/(H₂ + butanol) | 1.03 | 0.98 | 1.05 | 1.0 |
| Conversion, % | 18 | 52 | 54 | 48 |
| decarboxylation | 90 | 94 | 94 | 96 |
| C3 purity, % | 76 | 70 | 78 | 85 |
| Iso-butanol | <0.5 | 2 | 1 | 1 |
| butanoic acid | 10 | 2 | 4 | 3 |
| Heavy products | <0.5 | 2 | 1 | 0.2 |

Analysis of the products is performed by using an on-line gas chromatography. The GC analysis of the products show two major peaks corresponding to propylene and iso-butanal, and small peaks corresponding to unreacted iso-butanol, n-butanol, i-butanoic acid, heavy compounds. Di-butyl ether and iso-butyl iso butinate are also observed.

### Example 16 (Comparative)

Catalytic test was performed in the down flow stainless-steel reactor tube with an internal diameter of 11 mm. 2 g of crushed catalyst (35-45 mesh) was loaded. The reactor temperature was increased at a rate of 60° C./h to 450° C. under nitrogen flow 10 Nl/h. Then the catalyst was treated for 1 hour at 450° C. in nitrogen followed by reduction in di-hydrogen flow 10 Nl/min for 2 h at atmospheric pressure. Afterwards the reactor was purged with nitrogen followed by cooling down to the reaction temperature in nitrogen flow.

Analysis of the products is performed by using an online gas chromatography.

Catalyst A5 was used for the reaction.

The results are given in table 4.

TABLE 4

| | |
|---|---|
| T, ° C. | 325 |
| WHSV, h⁻¹ | 1.6 |
| Pressure, barg | 0.5 |

| | | | |
|---|---|---|---|
| feed | EFFLUENT | feed | EFFLUENT |

Composition, wt % C-basis

| | | | | |
|---|---|---|---|---|
| Decarboxylation (C3) | | 2.2 | | 3.6 |
| Dehydration (C4) | | 0.3 | | 0.5 |
| Iso-butanal | 25 | 21.9 | 50 | 44.1 |
| iBuOH | 75 | 64.9 | 50 | 47.3 |
| others | | 10.7 | | 4.4 |
| Conversion of iButanal, % | | 12.5 | | 11.9 |
| Conversion of i-BuOH, % | | 13.5 | | 5.4 |

Selectivity, wt % C-basis

| | | |
|---|---|---|
| Decarboxylation (C3=) | 8.5 | 20.9 |
| Dehydration (C4=) | 1.2 | 3.4 |
| others | 90.4 | 75.7 |

The data show that the catalyst modified with sulfur shows low activity in decarbonylation and the presence of the sodium (basic function) leads to high amount of heavies when both iBuOH and iButanal are present in the reaction mixture.

1.-15. (canceled)

16. A process for the conversion of primary C4 alcohol into propylene comprising:
contacting a stream (1) containing a primary C4 alcohol with at least one catalyst at a temperature ranging from 150° C. to 500° C. and at pressure ranging from 0.01 MPa to 10 MPa to transform the primary C4 alcohol into an effluent stream (2, 5) containing propylene, carbon monoxide and di-hydrogen, the transformation of primary C4 alcohol comprising at least a reaction of decarbonylation and optionally a decarboxylation reaction, the at least one catalyst comprising support which is non-acidic, having a TPD NH3 of less than 50 μmol/g and which is also a non-basic, having a TPD CO2 of less than 100 μmol/g.

17. The process according to claim 16 wherein stream (1) is contacted with the at least one catalyst to produce an effluent stream (2, 5) wherein at least 1 wt % of primary C4 alcohol is converted into propylene, carbon monoxide and di-hydrogen.

18. The process according to claim 16, wherein the step of contacting the primary C4 alcohol stream (1) with the at least one catalyst is performed in a single reaction zone (A) and the at least one catalyst is submitted before the transformation of primary C4 alcohol to a reduction in presence of di-hydrogen (H₂) at a temperature of at least 400° C. for at least 1 hour, at least one diluent being optionally added to stream (1), and optionally the diluent is recovered in the effluent stream (2) and optionally recycled in stream (1).

19. The process according to claim 16 wherein
stream (1) is contacted with at least one first catalyst comprising a support which is non-acidic, having a TPD NH3 of less than 50 μmol/g and which is also a non-basic, having a TPD CO2 of less than 100 μmol/g in a first reaction zone (B) at a temperature ranging from 150° C. to 500° C. and at pressure ranging from 0.01 MPa to 10 MPa to produce a stream (3) in which at least 1 wt % of primary C4 alcohol is converted into butyraldehyde, at least one diluent being optionally added to the stream (1),
optionally separating unconverted compounds from stream (3) and optionally the diluent if any and recycling unconverted compounds and optionally the diluent if any into stream (1) to obtain a purified stream (4),
contacting stream (3) and/or optionally the purified stream (4) with at least one second catalyst comprising a support which is non-acidic, having a TPD NH3 of less than 50 μmol/g and which is also a non-basic, having a TPD CO2 of less than 100 μmol/g, in a second reaction zone (C) at a temperature ranging from 150° C. to 500° C. and at pressure ranging from 0.01 MPa to 10 MPa to produce an effluent stream (5) comprising propylene, carbon monoxide and di-hydrogen, the second non-acidic catalyst, being optionally reduced, before the transformation of stream (3) and/or stream (4), in presence of di-hydrogen at a temperature of at least 400° C. for at least 1 hour,
optionally separating unconverted compounds and optionally the diluent if any from effluent stream (5) and recycling unconverted compounds into purified stream (4) and/or in stream (1), optionally recycling the diluent if any in stream (1) and/or stream (3).

20. The process according to claim 19, wherein the first catalyst and the second catalyst is the same catalyst comprising a non-acidic support having a TPD NH3 of less than 50 and a non-basic support having a TPD CO2 of less than 100 μmol/g.

21. The process according to claim 19, wherein the stream (3) and/or optionally the purified stream (4) treated in the second reaction zone (C) present a content of unconverted compounds of less than 10 wt %.

22. The process according to claim 18, wherein the reaction zones (A), (B), (C) are each operated under a temperature ranging ranging from 200° C. to 450° C., and under pressure ranging from 0.1 MPa to 3.5 MPa.

23. The process according to claim 19, wherein reaction zone (B) is operated under a temperature ranging from 150° C. to 450° C., and under partial pressure of butanols ranging from 0.01 MPa to 2.5 MPa and wherein reaction zone (C) is operated at a temperature ranging from 150° C. to 500° C., and under partial pressure of butyraldehyde ranging from 0.01 MPa to 1 MPa.

24. The process according to claim 16, wherein the catalyst, is chosen from:
a non-acidic and non-basic catalyst comprising a support which is non-acidic and non-basic, and optionally at least one metal dispersed on the at least one support at a content of at least 0.05 wt % based on the weight of the catalyst;
a non-acidic and non-basic catalyst consisting of at least one support being metal in a metallic state, in an oxidized state or in a partially reduced oxide state or
optionally the at least one metal is a transition metal chosen in the groups IB, IIB, IVB, VB, VIB, VIIIB, preferably at least one metal is chosen in the groups IIIA and IVA or chosen among palladium and/or platinum.

25. The process according to claim 24, wherein the non-acidic and non-basic catalyst comprises two metals chosen in the groups IB, IVB, VB, VIB, VIIIB, IIIA, IVA.

26. The process according to claim 24, wherein the at least one non-acidic and optionally non-basic support is selected from passivated alumina, activated carbon, metal silicate, perovskytes, silica-magnesia, phalocianides, silica, ceria, zirconia, titania, clays.

27. The process according to claim 16, wherein the at least one non-acidic and optionally non-basic catalyst contains one or several elements chosen from Na, K, P, B, S, each at a content of less than 2 wt % based on the weight of the catalyst.

28. The process according to claim 16 wherein secondary products formed via dehydration of the primary C4 alcohol are separated and cracked in lower olefins in a dedicated olefin cracking reaction zone or recycled back to stream (1).

29. The process according to claim 16 wherein the primary C4 alcohol is originated from a bio source optionally via a fermentation route or is originated from a mixture of heavy alcohols synthesis, which was produced via syn-gas routes.

30. The process according to claim 16 wherein the carbon monoxide and di-hydrogen are separated from stream (2, 5) and are subsequently used in at least one of the following processes:
transformation to liquid products via fermentation route,
production of alcohols from C1 to C5, such as methanol via syn-gas conversion routes,
production of hydrocarbons via syn-gas conversion routes including Fischer-Tropsch synthesis, olefins synthesis, aromatics synthesis or any combination of thereof,
hydro oligomerization of ethylene,
selective transformation of the ethylene to propanal, acetone, (n-, i-) propanols and propylene.
